(19) United States
(12) Patent Application Publication
(10) Pub. No.: US 2010/0000523 A1
(43) Pub. Date: Jan. 7, 2010

(54) Unichamber

(76) Inventor: Melvin S. Rosh, New York, NY (US)

Correspondence Address: MELVIN S. ROSH, 150 E. 93 STREET, APT. 8-C, NEW YORK, NY, 10128, US

(21) Appl. No.: 12/217,490

(22) Filed: Jul. 7, 2008

(51) Int. Cl. A61M 15/00

(52) U.S. Cl. 128/200.22

(57) ABSTRACT

The UNICHAMBER is a small and efficient “spacer” devise used to efficiently deliver aerosol medication to the lung obstructed patient. A wing-like projection within the bottom chamber indicates when the canister of medication is empty or when the patient is not taking the proper inhalation. The parts are easily dismantled for cleaning or replacement.

### BACKGROUND

More than three million Americans suffer with a medical condition known as ASTHMA and millions more suffer with CHRONIC OBSTRUCTIVE LUNG DISEASE. Both diseases are characterized by wheezing, shortness of breath and coughing. Numerous medications have been formulated to treat the conditions.

The most popular form of treatment is inhaled medication via a metered dose inhalation (MDI) canister. When the canister is activated during inspiration, medication propels into the throat at high speeds. Much of the propelled medication is lost as it bounces off the posterior pharyngeal wall and is propelled out of the mouth. To overcome this problem, the medication can be expelled into a chamber, known as a “spacer”, and then inhaled into the lungs.

CURRENT US CLASS: FIELD OF SEARCH

| | | |
|---|---|---|
| 5,425,089 | Jun. 27, 1995 | Kraemer |
| 6,470,882 | Sep. 29, 1997 | Newhouse |
| 5,792,057 | Aug. 11, 1998 | Rubsamen |
| 6,202,643 | Mar. 20, 2001 | Sladek |
| 6,039,042 | Mar. 21, 2001 | Sladek |
| 6,425,392 | Jul. 30, 2002 | Sosiak |
| 6,550,473 | Apr. 22, 2003 | Sladek |
| 6,584,969 | Jul. 1, 2003 | Farmer |
| 6,679,252 | Jan. 20, 2004 | Sladek |

### BRIEF SUMMARY OF THE INVENTION

Inhalation medications are best administered by means of a “spacer” devise. The use of a spacer makes delivery of medication far more efficient. The Unichamber is a small spacer, approximately 4×2 inches, that is conveniently carried in a pocketbook, a briefcase or in a belt pouch and allows the patients to have MDI medication at their disposal when needed.

The Unichamber consists of a lower and upper chamber. Attached to the upper chamber is the mouthpiece, which when not is use is covered by a removable cap. Within the lower chamber is the canister holder, which holds a MDI canister of medication. When pushed, the canister releases medication through the holder nozzle into the lower chamber. The canister holder is opened at the base in which the MDI canister is placed. Next to the canister holder is the air intake tube with two lateral side openings or windows. A latex-free rubber sleeve with a wing projection slips over the air intake tube. Rubber flaps on the opposite sides of the rubber sleeve fit over the windows of the air intake tube.

As the medication particles spray into the lower chamber of the spacer, the latex-free rubber valves closes the opening on the air intake tube, preventing loss of medication, and the latex-rubber wing projection is pushed upward, thus disseminating the particles of medication and indicating by its movement that the canister has discharged. If the canister is empty, the wing projection will not move. Likewise, if the patient is not taking in a deep enough inspiration, the wing projection will not move. The diaphragm, with its slit valve, between the two chambers also prevents most the medication from escaping the lower chamber.

The patient is instructed to take three deep breaths before using the Unichamber. After the last deep expiration, to hold the breath and place his or her mouth over the mouthpiece of the spacer devise. The patient, without removing the lips from the mouthpiece, depresses the MDI canister, takes in three deep breaths; exhaling through his or her nostrils or through the lateral sides of his or her mouth around the mouthpiece. As the breath is inhaled, the rubber valves on the window of the air intake tube opens to allow air to rush into the spacer and pulls the air through the diaphragm valve between the chambers into the lungs. Following inhalation, while the patient is exhaling through the nostrils or sides of the mouth, the rubber side flaps on the central tube acts as a valve and closes the windows of the air intake tube, preventing loss of medication. After three breaths, most of the medication in the chambers will have been delivered into the pulmonary system. The Unichamber is ready for the administration of the next dose of medication.

The upper and lower chambers can be separated for cleansing the parts of the Unichamber. When together, a latex-free rubber gasket seals them for prevention of loss of medication from the chambers.

### BRIEF DESCRIPTION OF THE DRAWINGS

FIG. 1: Unichamber

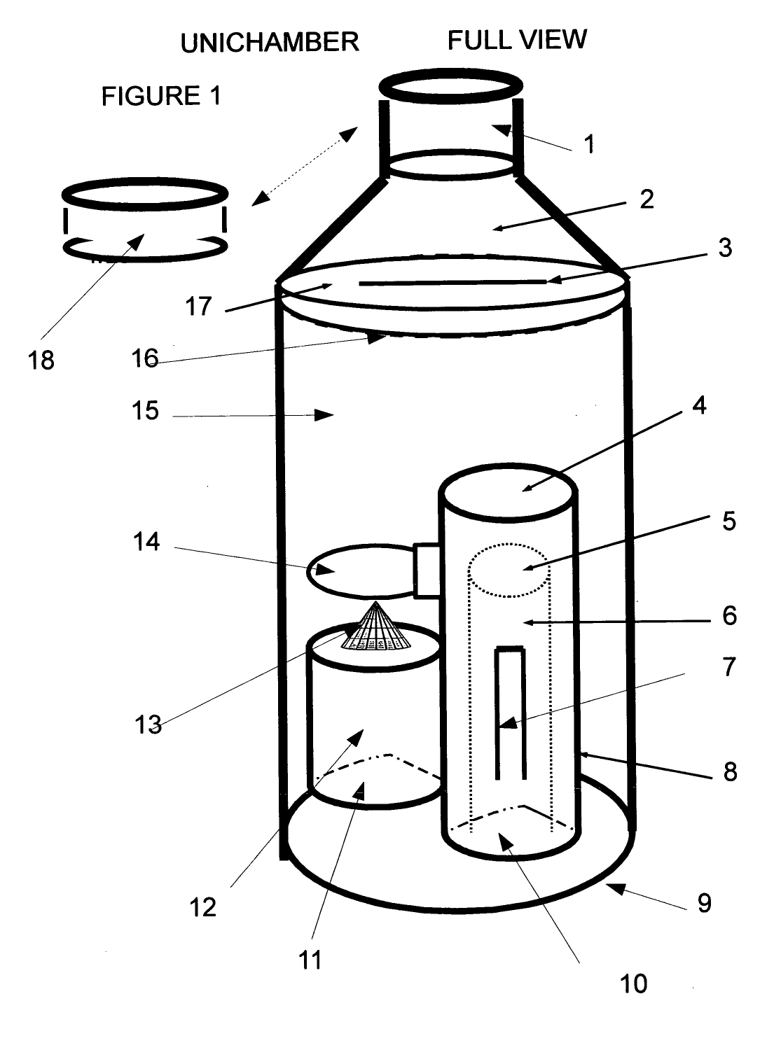

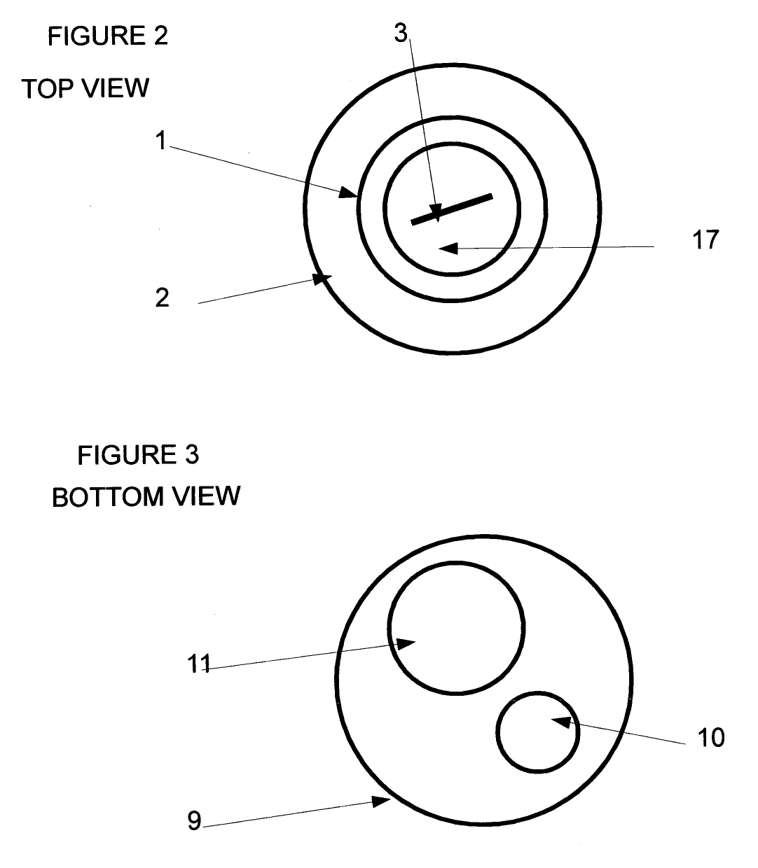

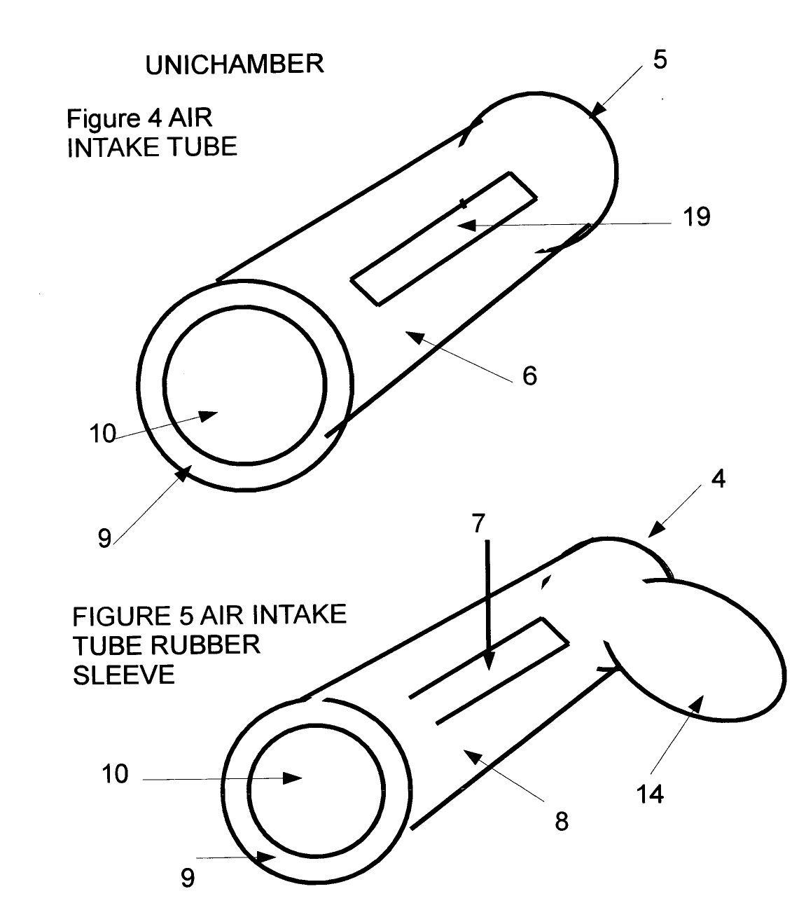

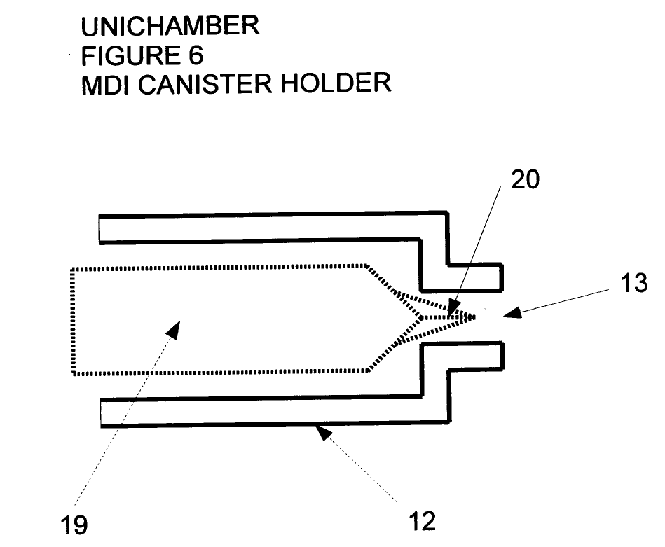

Displays the Unichamber with a metered dose inhaler (MDI) canister holder (12). The canister (not shown) fits through the canister opening(11) into the MDI canister holder (12) in the base (9) of the Unichamber.

The Unichamber consists of two parts, a bottom chamber(15) and a top chamber(2). Both chambers fit tightly together and are sealed with a latex-free rubber gasket(16). The bottom and top chambers can be easily separated for cleaning. The oval mouthpiece(1) is at the head of the top chamber(2). A removable cap (18) seals the mouthpiece(1) closed when the Unichamber is not in use.

A latex free rubber diaphragm (17) with a movable slit-valve opening (3) is situated between the bottom chamber(15) and upper chamber (2). The slit-valve (3) helps keep most of the medication in the bottom chamber of the Unichamber, and will freely open when an inhalation is taken by the patient, allowing medication to reach the lungs of the patient.

The air intake tube rubber sleeve (8) fits over the air intake tube(6) and sits along side the canister holder (12). The air intake tube rubber sleeve (8) has a wing-like latex projection (14) extending from its side and is situated over the canister holder nozzle (13). Two windows on the opposite sides of the air intake tube (6) are covered by two flaps (7) on either side of the air intake tube rubber sleeve (8) and control the flow of air through the windows. The bottom of the air intake tube (6) has an opening (10) which extends through the base (9) The upper ends of the air intake tube(5) and the upper end of the rubber sleeve(4) are sealed.

FIG. 2: Air Intake Tube—Top View

The mouthpiece(1) of the Unichamber is seen from the top. Through the opening of the mouthpiece a latex free diaphragm(17) with a slit valve(3) can be observed.

FIG. 3: Air Intake Tube—Bottom View

Two openings can be seen from the base(9) of the Unichamber. The larger is the opening(11) for the MDI canister holder (12). The smaller opening(10) is for the air intake tube (6).

FIG. 4: Air Intake Tube—Lateral View

An open window (19) is placed on opposite sides of the air intake tube (6). The top of the air intake tube (5) is sealed. The bottom opening (10) of the air intake tube (6) is in the base (9) of the Unichamber.

FIG. 5: Rubber Sleeve with Wing

Over the air intake tube(6) fits a removable latex-free rubber sleeve(8) with a movable slide flap(7), which cover the open windows(19) of the air intake tube(6) and act as valves. A movable latex-free wing(14) projects from the upper end of the rubber sleeve(8). The top of the latex sleeve (4) is sealed. The bottom is attached to the base (9) and is open (10).

FIG. 6: MDI Canister Holder

A MDI Canister(19) is shown in the canister holder(12). When the canister is pushed into the canister holder(12), the nozzle of the canister (20) is depressed and a dose of medication is pushed out of the spray opening(13) into the bottom chamber (15) of the Unichamber.

### DETAILED DESCRIPTION OF THE INVENTION

The Unichamber is composed of three parts: a bottom chamber(15), a top chamber(2) which leads into the mouthpiece(1) and a mouthpiece cover(18). The base of the bottom chamber(15) has two openings. The top opening(10) leads into the air intake tube(6). The other and larger opening (11) leads into the canister holder(12) for an MDI canister of inhalation medication.

When the canister is pushed down into the canister holder(12), medication is released into the bottom chamber, at which point, the canister is ready for the next dose of medication. The Unichamber can be easily taken apart for cleaning.

The air intake tube(6) has two open windows(19), one on each lateral side of the air intake tube(6). The upper end of the air intake tube(4) is sealed; the lower open end is attached to the base(9) of the bottom chamber(15).

The latex-free rubber sleeve(8), slides over the air intake tube(6). The sleeve has two side flaps(7) which fit over the open windows(19) of the air intaketube(6). When the MDI canister injects medication into the spacer, the side flaps(7) close the windows(19) of the air intake tube(6). Upon inspiration by the patient, the side flaps(7) open the windows(19), allowing air to come into the spacer. At the upper end of the rubber sleeve(8) is a rubber “wing” projection(14), which moves when medication is released from the MDI canister or when the patient takes an inspiration. The rubber sleeve(8) is removable for cleaning or replacing.

A MDI canister(19) is placed into the canister holder(12). When the MDI canister(19) is pushed into the holder(12), the nozzle(20) is depressed, releasing aerosol medication through the spray opening(13) into the bottom chamber of the spacer. The canister holder(12) is made to fit a standard MDI canister(19).

The assembled UNICHAMBER is approximately four inches in length and two inches in width. It can be easily carried in a pocketbook or in a belt pouch. Latex-free rubber gaskets(16) seal the top and bottom chambers.

I claim:

1. A Unichamber is a small medication inhalation apparatus (spacer) which is used to facilitate the inhalation of most of the medications dispensed by a MDI canister.

2. The propelled particles from the MDI canister is dispersed throughout the chamber of the spacer as they contact the latex-free rubber wing-like projection attached to the latex-free rubber sleeve on the air intake tube; movement of the rubber wing-like projection indicate release of medication from the MDI canister and proper inhalation by the user.

3. Latex free rubber valves on the air intake tube control the direction of air flow into the chamber.
